(19) United States
(12) Patent Application Publication
(10) Pub. No.: US 2011/0045581 A1
(43) Pub. Date: Feb. 24, 2011

(54) BIOREACTOR FOR CONTINUOUS PRODUCTION OF BIOLEACHING SOLUTIONS FOR INOCULATION AND IRRIGATION OF SULFIDE-ORE BIOLEACHING HEAPS AND DUMPS

(75) Inventors: Roberto Collao Olivares, Providencia (CL); Leandro Mauricio Padilla Iglesias, Providencia (CL); Ricardo Badilla Ohlbaum, La Reina (CL)

Correspondence Address: MERCHANT & GOULD PC, P.O. BOX 2903, MINNEAPOLIS, MN, 55402-0903, US

(73) Assignee: BIOSIGMA S.A., Colina (CL)

(21) Appl. No.: 12/859,083

(22) Filed: Aug. 18, 2010

(30) Foreign Application Priority Data
Aug. 20, 2009 (CL) 1749-2009

(51) Int. Cl. C12M 1/00

(52) U.S. Cl. 435/293.1

(57) ABSTRACT

The invention discloses an air-lift bioreactor, with internal recirculation, for producing sulfide-ore and minerals bioleaching solutions, with a phase-separating and solids-recirculation system without needing to impel the suspension containing the solids to the bioreactor by means of pumps, using diatomaceous earth and/or ferric precipitates as solid support to immobilize iron and sulfur-oxidizing microorganisms. Specifically speaking, the invention describes a bioreactor that continuously produces bioleaching solutions containing microorganisms for inoculation and irrigation of sulfide-ore heaps and dumps processed by bioleaching. The bioreactor is stirred pneumatically, and is generally made up of an air diffuser, a reaction zone, a de-gasification zone, a solids separation zone, a culture media inlet, and a bioleaching solution outlet. Depending on the source of energy supplied for the growth of microorganisms, the bioreactor can produce a solution concentrated in ferric ions, iron-oxidizing bacteria and reduced-sulfur-compound-oxidizing bacteria.

This application claims benefit of Serial No. 1749-2009, filed 20 Aug. 2009 in Chile and which application is incorporated herein by reference. To the extent appropriate, a claim of priority is made to the above disclosed application.

### SCOPE OF THE INVENTION

The invention is linked to the field of ore bio-leaching and discloses an air-lift bio-reactor with internal recirculation for producing sulfide-ore bioleaching solutions, with a phase-separating and solids-recirculation system, without the need to impel the suspension containing the solids towards the bioreactor with pumps, using diatomaceous earth and/or ferric precipitates as a solid support to immobilize iron and/or sulfur-oxidizing microorganisms. Specifically speaking, the invention describes a bioreactor that continuously produces bioleaching solutions containing microorganisms, for inoculation and irrigation of heaps and dumps of sulfide ores processed by bioleaching. The bioreactor is stirred pneumatically, and is generally made up of an air diffuser, a reaction zone, a degasification zone, a solids-separation zone, a culture media inlet, and a bioleaching solution output zone. Depending on the source of energy supplied for the growth of the microorganisms, the bioreactor can produce a solution concentrated in ferric ions, iron-oxidizing bacteria and reduced-sulfur-compound oxidizing bacteria.

### BACKGROUND INFORMATION OF THE INVENTION

Over 90% of the world's mine copper is currently obtained from copper sulfide ore processing. Among the copper sulfide species present in the ores, the most important are chalcopyrite, bornite, chalcosite, coveline, tenantite and enargite, of which chalcopyrite is the species in greatest relative abundance, and therefore the one of most financial interest.

Copper sulfide ore processing is currently sustained by technologies based on physical and chemical processes associated with crushing, grinding, and flotation of ores, followed by fusion-conversion of the concentrates and electrolytic refining of the metal. Practically speaking, over 80% of copper is produced by processing ores following the described route—known as conventional—which is limited to medium and high-grade ores, according to the specific characteristics of the deposits and ore-processing plants. Because of this, there are vast and valuable relatively low-grade mineral resources which are sub-economical with conventional techniques, and remain unexplored for lack of effective technology for their exploitation.

On the other hand, ores in which copper is present in the form of easily soluble in acid oxidized species, are processed by means of acid-leaching processes, followed by solvent-extraction and metal-electrowinning processes, in what is known as the hydrometallurgical route for copper recovery. This route is very attractive because of its lower operation and investment cost compared to conventional technologies, as well as because of its lower environmental impact. Nevertheless, applications of this technology are limited to oxide ores and to certain copper sulfide ores (chalcosite, coveline and bornite). In this last case, the metal is soluble in acid in the presence of an energetic oxidizing agent catalyzed by microorganisms (Uhrie, J L, Wilton, L E, Rood, E A, Parker, D B, Griffin, J B and Lamana, J R, 2003, “The metallurgical development of the Morenci MFL Project”, Copper 2003 Int Conference Proceedings, Santiago, Chile, Vol VI, 29-39).

It has been established for a long time that solubilization or leaching of sulfide ores is aided by the presence of iron and sulfur-oxidizing bacteria, known as bioleaching. When these ores are worked through commercial-scale leaching in heaps or dumps, using mesophilic microorganisms in the range of 25-45° C., satisfactory recovery and extraction rates—of 80% recovery over a period of approximately one year's operation—are achieved for the bioleaching of secondary sulfides, such as coveline (CuS) and chalcosite (Cu₂S). Within this temperature range, the most widely described microorganisms present correspond to bacteria of the Acidithiobacillusand Leptospirillum genre, of which, the most common species are Acidithiobacillus ferrooxidans, Acidithiobacillus thiooxidans, Leptospirillum ferriphilum and Leptospirillum ferrooxidans.

According to the above, various processes seek the way to promote growth conditions for the microorganisms participating in the bioleaching. For example, patent application WO2004/027100 introduces a method in which microorganisms, free of their exopolymers, are produced, to subsequently be injected into a bioleaching heap in which they are provided with the nutrients and/or the conditions necessary for them to generate these exopolymers. Patent application WO00/71763 proposes introducing acid liquor containing bacteria into the bioleaching heap. Patent application U.S. 2004/0091984 mentions the incorporation of bacterial cultures obtained from leaching ponds, to promote bioleaching.

Although the previously mentioned documents mention the incorporation of microorganisms into bioleaching heaps, no references to, or descriptions of reactors propounded as necessary for microorganism culture, are found.

As we can observe, based on the documents quoted, there is great concern regarding the increase in the number of active microorganisms in ores, in order to promote bioleaching, and particularly, regarding the increase of a certain type of microorganisms, a type that depends on the bioleaching carried out. This can be explained with two reasons:

Firstly, the native microorganisms present in the ore or their growth kinetics, may not be the most appropriate for the bioleaching conditions employed in the process, which explains the inoculation of specific microorganisms.

Secondly, starting the process of bacterial bioleaching of copper sulfides requires the bacteria to come into contact with the surface of the ore to be bioleached, and then multiply so as to colonize the surface of the available solid. Once this colonization has occurred, bioleaching kinetics grows faster (Lizama, H. M., Fairweather, M. J., Dai, Z., Allegretto, T. D. 2003. “How does bioleaching start?”. Hydrometallurgy. 69: 109-116). In this sense, a latency phase or so-called “lag phase” during which the dissolution kinetics of the ore is slow, has been observed in bioleaching pilot operations, a fact that has been associated with the phase in which microorganisms colonize the ore surface (Lizama, H M; Harlamovs, J R; Belanger, S; Brienne, S H. 2003. “The Teck Cominco HydroZinc process”. Hydrometallurgy 2003: 5th International Symposium Honouring Professor Ian M. Ritchie; Vancouver, B C; Canada; 24-27 Aug. 2003. pp. 1503-1516).

Therefore, if provided with a bioreactor that allows the large-scale culture and/or propagation of microorganisms for inoculation in economic terms, it would be possible to shorten the duration of the phase in which the ore is colonized by bacteria which in turn equals a reduction of the total bioleaching time, and also to achieve a high concentration of bioleaching bacteria on the ore surface, leading to faster bioleaching of the ore.

From the point of view of the underlying biology, it is known that the growth of bioleaching microorganisms is sensitive to parameters such as temperature, pH, the composition of the solution, aeration, among others, over which there is little control in a heap or dump and which furthermore vary widely during the working time of these systems, as well as with the location within the system, and can therefore be far from the optimum conditions it is possible to achieve in a bioreactor in which there is more control over these parameters, because of which the inoculation of heaps and dumps with leaching microorganisms produced in reactors under controlled conditions, in batch or continuous mode, proves to be of interest.

As we can observe, the industrial practice of bioleaching operations in heaps and dumps does not consider the controlled production of microorganisms useful in this bioleaching at a scale fitted to the problem, microorganisms that could be advantageously employed to reduce the ore colonization phase, or to increase the concentration of microorganisms in this ore. Therefore, as far as we know, we can state that the need for a culture system, such as for example a bioreactor with controlled conditions that allows the large-scale continuous culture and/or propagation of microorganisms useful in ore bioleaching persists. Apart from making the controlled and continuous production of microorganisms that are useful in ore bioleaching possible, the reactors in which this process is carried out must have high volumetric productivity (concentration of cells in effluent/culture residence time in the reactor). This requirement becomes evident if we consider that in hydrometallurgy, particularly in that of copper, the flows of treated ore are considerably large. The consequence of this would be the need to produce large flows of bioleaching solution to inoculate the ore of the heaps. If the type of reactors used for this purpose has low productivity, the resulting reactors will be considerably high, and as a consequence, investment and operation costs will be very high as well.

A technology that is promising for increasing the volumetric productivity of bioreactors operated in continuous mode consists in the culture of cells immobilized on biofilms on solid supports. It happens that the minimum hydraulic residence time required for a given conversion in reactors operated in continuous mode, without immobilized biomass, is limited by the magnitude of the specific growth rate of the microorganism used, a parameter that cannot be arbitrarily increased. The operation of a continuous bioreactor with lower than minimum hydraulic residence time, will inevitably lead to “bioreactor washout”, a condition in which the biomass present in the bioreactor disappears because the growth rate is insufficient to compensate the dilution of the microorganisms caused by the flow of solutions fed through the reactor during the continuous operation. Typical values found for residence time in stirred reactors for the growth of extremophilic microorganisms such as those used in ore bioleaching, are within the range of 48 to 24 hours (P. d'Hugues, C. Joulian, P. Spolaore, C. Michel, F. Garrido, D. Morin. 2008. “Continuous bioleaching of a pyrite concentrate in stirred reactors: Population dynamics and exopolysaccharide production vs. bioleaching performance “Hydrometallurgy. 94: 34-41)

A way to overcome this limitation is to promote the fixing of microorganisms within the reactor, known as wall effect. In reactors with cells immobilized in biofilms on solid supports and operated continuously, as the quantity of microorganisms immobilized in the reactor increases, it is possible to operate with residence times lower than the ones that can be achieved with conventional technology because of the increase in active biomass as compared to without immobilized cells, which makes it possible to reduce the size of the bioreactor required for a given biotransformation, or what equals the same, increase its volumetric productivity.

Immobilization of microorganisms in microbial biofilms in reactors has multiple applications in industrial processes in areas as diverse as plant-cell culture (Archambault, J., Volesky, B., Kurz, W. G. 1989. “Surface immobilization of plant cells”. Biotechnology and Bioengineering. 33: 293-299), waste-water treatment (patent application WO1993/025485A1) and the production of organic compounds (Qureshi, N., Annous, B. A., Ezeji, T. C., Karcher, P., Maddox, I. S. 2005. “Biofilm reactors for industrial bioconversion processes: employing potential of enhanced reaction rates”. Microbial Cell Factories. 4: 24.). The extent of biofilm development depends on the surface available for colonization, and a way of increasing the wall effect is to add a finely divided solid with high surface density to the reactor. For example, diatomaceous earth and activated carbon in bioreactors have been used to this end (Van der Meer, T., Kinnunen, P. H. -M., Kaksonen, A. H., Puhakka, J. A. 2007.“Effect of fluidized-bed carrier material on biological ferric sulphate generation”. Minerals Engineering. 20: 782-792).

In order to improve mass transference within the system and reduce shear stress in the fluid, reactors with immobilized biomass used in the industry are of the fluidized-bed and air-lift type. Air-lift reactors are characterized in that they have a central tube which allows two volumes inside the same reactor to be separated, one in which there is an ascending column of liquid and air (raiser), and another descending one of liquid (down-comer). The ascending flow in the raiser is generated by injection of air through the bottom of the reactor. An important advantage of air-lift reactors regarding other pneumatically stirred reactors is a lower gas-velocity requirement (and therefore, lower power consumption) to keep the solid particles in suspension (Heck, J. and Onken, U. 1987.“Hysteresis effects in suspended solid particles in bubble columns with and without draft tube”. Chemical Engineering Science. 42: 1211-1212; Becker, R. -J., Hiippe, P., Wagner, K. and Hempel, D. C. 1987.“Einsatz eines Suspensions-Air-lift-Schlaufenreaktors zur Reinigung problematischer Abwässer”. Chem. Ing. Tech. 59: 486-489).

For an airlift reactor to be able to operate with solids in suspension, the installation of settling systems in the equipment, to make removal of the solid from the outgoing flow possible, has been described. The design of this equipment includes the forced return of the settled solids to the reaction zone (Mulder, R. M., Vellinga, S. H. J. 1996. “System and process for purifying waste water which contains nitrogenous compounds”. U.S. Pat. No. 5,518,618). This design considers the use of a pump to impel the suspension of solids at the bottom of a first settler, from a second jacket-shaped settler that covers most of the external mantle of the first settler.

The objective of this invention is specifically to provide a reactor for the continuous production of bioleaching solutions with high concentrations of microorganisms and ferric ions, to inoculate and enrich solutions used for irrigating sulfide ore heaps and dumps. The bioleaching solutions produced by this bioreactor consist of concentrated solutions of iron-oxidizing bacteria, reduced-sulfur oxidizing bacteria, ferric ions, or some mix of the above. The bacteria produced and used can be selected strains, or consortiums of native microorganisms from the ore we wish to bioleach.

The bioreactor of this invention has high productivity due to the use of bacteria immobilized on diatomaceous earth or on ferric ion precipitates generated during the period of growth of iron-oxidizing bacteria in the reactor and to the presence of a device that is able to retain the solid support with immobilized microorganisms in the reactor, which does not require pumps to impel the settled suspension, and whose separator is located in the upper part of the reactor, greatly aiding operation and maintenance when compared to the one described in previously mentioned U.S. Pat. No. 5,518,618.

With the bioreactor of the present invention, it has been possible to resolve a problem of the art, because with it, is possible to obtain bioleaching solutions with high concentrations of biomass, in the order of 10⁸ to 10⁹ bacteria/mL and high concentrations of ferric ions of up to 25 g/L applied in the inoculation and irrigation of sulfide-ore heaps and dumps processed through bioleaching.

In order to have a better understanding of the processes linked to the continuous and controlled generation of inoculum, the following concepts are to be understood:

Bioleaching microorganism: microorganism capable of promoting, because of its metabolic activity, the solubilization of metallic ions from oxide and sulfide metallic species.

Energy source: Compound or element used by microorganisms as a source of energy for growth. In the case of leaching microorganisms, typical sources of energy are the ferrous ion (Fe(II)), elemental sulfur, and reduced forms of elemental sulfur (sulfide, thiosulfate and tetrathionate). According to the preference for a specific energy source it is possible to establish a classification of leaching microorganisms: i) iron oxidizing or ferrooxidizing microorganisms whose source of energy is ferrous ions, and; ii) sulfur-oxidizing or thiooxidant or sulfooxidant microorganisms, whose source of energy is elemental sulfur and/or reduced sulfur species.

Culture media: Aqueous media containing the energy source and the nutrients (ammonium salts, phosphate, and magnesium) required for microbial growth.

Batch operation: Type of culture in which there is no exchange of solution between the reactor and the exterior, excepting equipment loading and unloading stages. The propagation of microorganisms from inoculums is achieved during this stage.

Continuous operation: Type of culture in which an output flow of culture from the reactor exists simultaneously with an input flow of culture-media to the reactor. This type of operation allows continuous generation of bacteria inoculums with which heaps and/or dumps are irrigated.

In order to be supplied with a large quantity of microorganisms capable of leaching sulfide metallic ores using bioreactors and controlled conditions, a reactor that allows the large-scale propagation of biomass that can be used in bioleaching of sulfide metallic species, has been developed. This reactor is a particular bioreactor that allows the continuous production of microorganisms of different types, such as, Acidithiobacillus ferrooxidans, Leptospirillum ferrooxidans, Leptospirillum ferriphilum, Ferroplasma sp. and Acidithiobacillus thiooxidans, separated or together, with or without native microorganisms.

### BRIEF DESCRIPTION OF DRAWINGS

FIG. 1 schematically illustrates the bioreactor according to the present invention.

FIG. 2 schematically illustrates the air injection devices of the bioreactor of the present invention.

FIG. 3 schematically illustrates a cross cut of a diffuser of the bioreactor's air injection devices according to the present invention.

FIG. 4 is a graph of the total concentration of iron in the feed (A1) and effluent (E1))of the bioreactor throughout the duration of a bioreactor test.

FIG. 5 is a graph of the concentrations of ferric iron in the feed streams (A2) and effluent (E2) of the bioreactor throughout the duration of the execution of the test.

FIG. 6 is a graph illustrating the productivity of iron (III) throughout the duration of the test.

FIG. 7 is a graph of the concentration of free biomass in the reaction zone (B) and in the effluent (E3) of the bioreactor throughout the duration of the test.

FIG. 8 is a graph of the free biomass productivity of the reactor throughout the duration of the test.

FIG. 9 is a graph of the concentrations of solids (C_{P} ) in the body (SB) and in the effluent (SE) of the bioreactor throughout the duration of the test.

### DETAILED DESCRIPTION

The present invention consists in a pneumatically stirred bioreactor of the air-lift type, with internal recirculation, for producing bioleaching solutions of sulfide ores, with a phase separator and recirculation of solids, using diatomaceous earth, sulfur and/or iron precipitates as a solid support to fix iron and/or sulfur oxidizing microorganisms.

The preferred arrangements of the present invention are described below with reference to the accompanying figures.

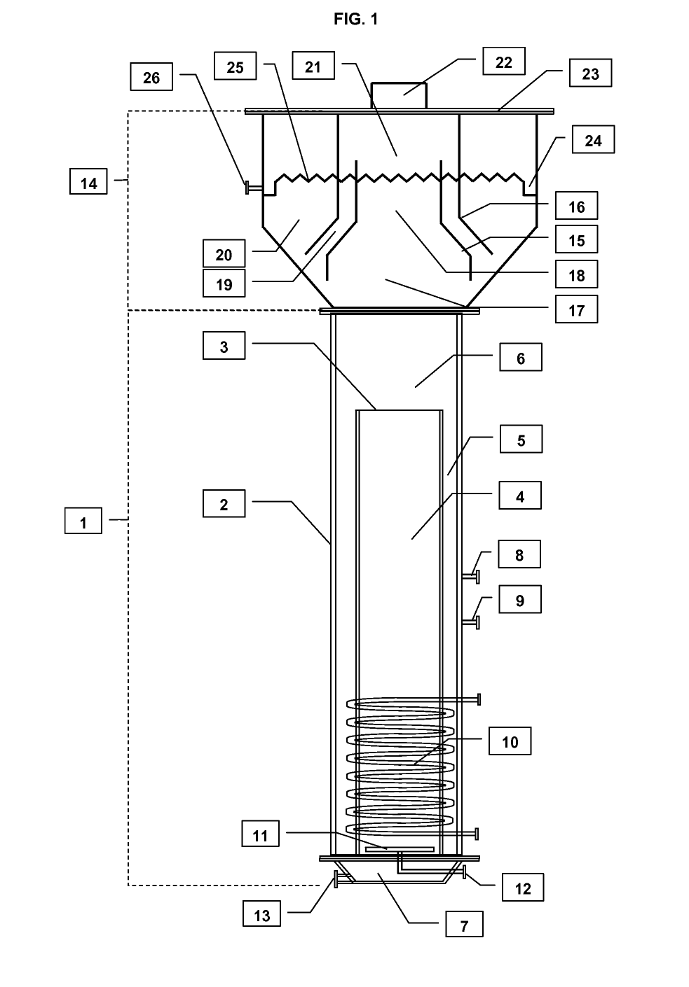

As illustrated in FIG. 1, a preferred ensemble of the present invention consists in a bioreactor that continuously produces bioleaching solutions with high concentrations of microorganisms and ferric ions, for the inoculation and irrigation of sulfide-ore bioleaching heaps and dumps, that essentially includes:
A reaction zone (1), that includes an external cylinder (2) and an internal cylinder (3) concentric to the external cylinder (2), which separates the reaction zone (1) into an ascending column (riser) (4) and a descending column (down-comer) (5), an upper recirculation zone (6) from the riser to the down-comer and a lower recirculation zone (7) from the down-comer to the riser;
an inlet for acid (8);
an inlet for culture media (9);
a heater (10) in the reaction zone (1) to keep the temperature within an adequate range for the growth of the microorganisms present;
air injection devices (11) in the lower part of the reaction zone (1), connected to the corresponding air input pipe (12) located at the lower part of the equipment.;
a discharge outlet (13) for quick emptying of the bioreactor in the lower recirculation zone (7);
a phase separator (14) that includes:
   a concentric internal separator part (15) that defines the input zone (17) in which the air bubbles are trapped and conducted in the three phase mixture towards the de-airing zone (21) which also defines the gaseous separation zone (18) for separating the air bubbles from the three phase mixture.
   a concentric external separator part (16) that defines the de-airing zone (21) on the inside; between the internal separator part (15) and the external separator part (16) which defines the zone of conduction (19) of the two phase mixture (solid-liquid) to the solids separation zone (20) and external side of the solids separation part zone (16) which defines the solids separation zone. an exit chimney (22) located in an upper covering (23) of the phase separator (14) and in fluid communication with the de-airing zone (21), a ring-shaped gutter (24) to form an accumulation zone for the clear liquid coming out of the bioreactor,
   a cylindrical wall with a dentate rim (25) abutted against the wall of the phase separator (14) marking the limits of the ring-shaped gutter (24), and an outlet (26) for gathering the solution concentrated in iron and/or sulfur oxidizing bacteria, and ferric ions.

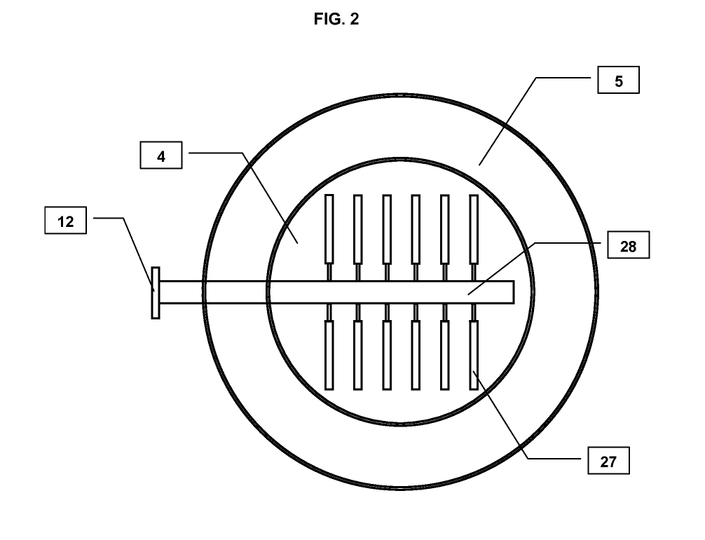

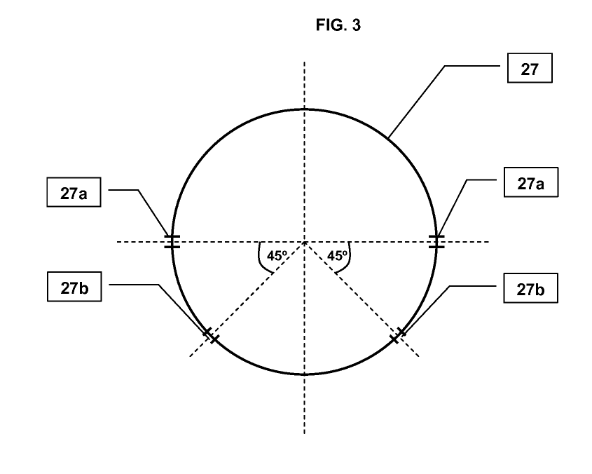

As illustrated in FIG. 2, the air-injecting devices (11) consist in a set of diffusers (27) of a perforated-pipe type, distributed at a short distance along the lower edge of the internal cylinder (3) separating the riser from the down-comer. The diffusers (27) are connected to an air-feeding main (28) with an air inlet (12). As you can see in FIG. 3, the vents (27a and 27b) of the air diffusers are located on both sides of the latter, at a maximum angle of 45° below horizontal. This allows the vents to be less prone to getting clogged up with the solid particles that serve as a support for the bacteria.

In the ensemble described, as a result of the injection of air at the base of the riser (4), a difference in hydrostatic pressure between the fluids in the riser (4) and in the down-comer (5) occurs, producing internal circulation of the culture media and the biomass-supporting particles, which allows optimum mixing and suspension of biomass-supporting solid particles in the reaction zone (1), and aids the transference of oxygen from the air to the microorganisms.

The tree phase mixture, gas-solid-liquid, coming from the reaction zone (1) reaches the phase separator (14), where, in the input zone, complete separation of the gaseous phase, which is discharged through the upper zone of the phase-separation zone (14), in the de-airing zone (21), through an exit chimney (22) occurs. The resulting degasified mixture enters the solids-separation phase (20), where the solid phase which includes the support particles with immobilized biomass, is completely separated from the degasified mixture and returned to the reaction zone (1). The design of the three phase separator for the phase separator (14), and the location of the diffusers (27) of the air-injection devices (11) allows efficient separation of the gaseous, solid and liquid phases in mixtures formed under conditions of high air-injection flow, operated with low residence times and with a high concentration of biomass-supporting particles. An important characteristic of the phase separator is that it allows fluid continuity between the settling zone and the reaction zone, making the use of recirculation pumps unnecessary.

During the continuous operation of the equipment, the feeding of culture media, that enters the bioreactor continuously, is carried out through the culture-media inlet (9), located in the mantle of the external cylinder (2) of the reaction zone (1), and comes into contact with the mixture descending through the down-comer (5). The acid inlet (8) for injecting concentrated acid if the pH requires controlling is also located in the mantle of the external cylinder (2). The mixture that descends through the down-comer (5) enters the riser (4) by the lower end of the reaction zone (1) through the lower recirculation zone (7), where the air injection devices (11) are located. Apart from supplying the oxygen and carbon dioxide required for the growth of the microorganisms, the injection of air also allows the tree phase mixture to ascend through the riser (4).

In order to keep the contents of the bioreactor at a temperature appropriate for the growth of the iron and/or sulfur oxidizing mesophilic bacteria, heat is added to the system in two different ways, depending on the operation mode of the bioreactor. During the batch operation, a heating device (10), such as for example a coil, located around the lower zone of the internal cylinder's mantle (3), makes it possible to heat the mixture descending through the down-comer (5). This way, heat lost through the walls and evaporation is compensated. During the period of continuous operation, a heat exchange element (not shown in the figures), such as for example a plate heat exchanger, is used to heat the culture media fed to the bioreactor through the culture media inlet (9); this way, heat lost through walls and evaporation, and through feeding a culture media at a temperature lower than that of the mixture contained in the reactor, is compensated.

Most of the three phase mixture ascending through the riser (4), returns to the down-comer (5) by the upper recirculation zone (6) of the reaction zone (1). Most of the bubbles follow their path from the riser (4) towards the input zone (17) at the lower end of the phase-separation zone (14), and only a fraction composed of smaller bubbles enters the down-comer (5). The phase separator (14) of the bioreactor of this invention includes, an internal separator part (15) shaped like a cylinder with a conic narrowing at the middle that defines the input zone (17) in its lower part and the gaseous separation zone (18) in its upper part. The lower part of the internal separator part (15) is wider than its upper part, and the diameter of the lower part of this internal separator part (15) is slightly larger than the diameter of the external cylinder (2) of the reaction zone (1). Thanks to this design, the internal separator part (15) is capable of concentrating most of the bubbles that ascend to the input zone (17), as well as the solid particles that are pulled along by the bubbles and serve as support for the growth of microorganisms. The gaseous separation zone, where most of the separation of the gaseous phase from the three phase mixture is carried out, is in the upper part of the internal separator part (18). The air that is separated from the three phase mixture is collected in the de-airing zone (21) space located above the internal separator part (15) and enclosed between the walls of an external separator part (16). Finally, the air collected in the de-airing zone (21) leaves the bioreactor of this invention through an exit chimney (22) located in an upper covering (23) of the bioreactor.

The external separator part (16) makes it possible to keep the stirring that occurs in the input zone (17) and in the gaseous separation zone (18) isolated, so that this stirring does not extend to the rest of the phase separator (14) where calm conditions are required in the mixture, particularly in the solids separation zone (20). There is a ring-shaped space that forms the conduction zone (19) between the internal separator part (15) and the external separator part (16), where separation of the air from the three phase-mixture continues to occur if this process has not been completed in the gaseous separation zone (18). Because calm conditions begin to predominate in the conduction zone (19) as it is descended through, the solid particles start to settle in this zone.

The lower part of the external separator part (16) has a conical shape that widens downward, and prolongs until it is at a short distance, between 4 and 8 cm, from the conical external wall of the lower segment of the phase separator (14). This design makes it possible to give the conduction zone (19) more length, and therefore to provide more residence time for separating the bubbles remaining in the mixture, as well as achieve settling of a significant fraction of solid particles. The upper part of the external separator part (16) is cylindrical and joined to the upper covering (23) of the bioreactor. The conditions of the mixture are even calmer in the solid-separation zone (20) and, considering the width and depth of this ring-shaped zone as well, settling of the rest of the solid particles occurs.

The phase separator (14) includes a cylindrically-shaped upper segment, and a conically-shaped lower segment that converges downward. The solid particles that settle in the conduction zone (19) and in the solids separation phase (20) descend over the internal surface of the conical wall of the lower segment of the phase separator (14), towards the upper recirculation zone (6). On the other hand, the clear liquid, free of air bubbles and solid particles, overflows onto a cylindrical wall with a dentate rim (25), and from there, passes into the ring-shaped gutter (24) that is placed against the separator wall. The clear liquid, in other words, the bioleaching solution, leaves the bioreactor from the ring-shaped gutter (24) through an outlet (26) in the wall of the phase separator (14).

The design of the internal separator part (15) and of the external separator part (16), of the phase separator (14) of the present invention is particularly appropriate for allowing settling of the solids used in this bioreactor that consist of particles of diatomaceous earth and iron precipitates, which tend to float very easily, and are therefore less susceptible to the settling process. On the other hand, the design of the internal separator part (15) makes efficient capturing of bubbles possible, considerably reducing the number of bubbles that escape into the solids settling zone.

The bioreactor of the present invention is initially loaded with particles of diatomaceous earth, pyrite, scavenger tails and/or sulfur that adhere to the microorganisms; we wish to produce, whether iron or sulfur oxidizing. As the microorganisms reproduce, a fraction remains fixed to the solid support, forming a biofilm, while another fraction passes into the liquid phase. In the case of iron-oxidizing organism production, as these organisms oxidize ferrous ions, the ferric ions that are generated begin to form precipitates (iron hydroxides and jarosite), the quantities of which depend principally on the pH of the three phase mixture and on the concentration of certain salts that encourage the formation of iron precipitates. As the process time elapses, it has been observed that the quantity of iron precipitate particles begins to increase, reaching concentrations which even surpass the concentrations of diatomaceous earth particles initially added to the bioreactor. Finally, the concentration of iron precipitates becomes stable with values that depend on the pH of the three phase mixture. The properties of iron precipitates as bacterial support particles are good, because they posses relatively high sedimentation rates that make their settling in the solids-separation zone easy, and are easily re-suspended, with a flow of air, from a settled state, without forming a strongly cohered mass. Nevertheless, in order to avoid excessive formation of iron precipitates, operating with pH values between 1.2 and 1.6 is recommended.

In the case of the production of sulfur-oxidizing microorganisms, these can be generated from some source of sulfur or its reduced species, as for example pulverized particles of elemental sulfur. Microbial oxidization of sulfur compounds generates sulfuric acid. Therefore, when iron and sulfur oxidizing microorganisms are produced jointly in a same bioreactor there is the advantage of saving acid because the acid required for oxidization of ferrous ions catalyzed by the iron oxidizing microorganisms is partly supplied by the acid generated by the sulfur-oxidizing microorganisms.

If elemental sulfur particles are used as a source of energy for the growth of sulfur-oxidizing microorganisms, these particles of sulfur can fulfill the role of solid support for the bacteria. Nevertheless, as the sulfur goes oxidizing, the size of these particles goes getting smaller. At some moment the particles reach a size with which the phase separator system is not capable of retaining them in the bioreactor, and they are therefore carried away by the bioleaching solution stream leaving the bioreactor. These particles can produce an obstruction in the liquid lines downstream in the process, including the droppers that distribute the bioleaching solution on ore heaps and dumps. To avoid the above from happening, the effluent solution of the present invention is made to pass through a filtering or settling system that allows the solution to be clarified. Another alternative is for the effluent solution of the bioreactor of this invention to pass into a second reactor provided with air injection, with residence time and air-flow sufficient to completely oxidize the remaining particles of sulfur.

The relationships fulfilled between the dimensions of the bioreactor parts according to the preferred arrangement of the present invention shown in FIG. 1 are indicated below.

The ratio between the transversal area of the riser (4) and the transversal area of the down-comer (5), with both areas free from the passage of flow, is approximately 1.0.

The ratio between the height H_{R} of the external cylinder (2) and its external diameter D_{R} is between 4 and 5.

The ratio between the height H_{R} of the external cylinder (2) and the height H_{D} of the internal cylinder (3) is approximately 1.22.

The height H_{F} of the lower recirculation zone (7) between the base of the bioreactor and the base of the internal cylinder (3), should be such that the transversal area of this lower recirculation zone (7) through which the mixture re-circulates from the down-comer (5) to the riser (4), is approximately equal to the transversal area of the down-comer (5) available to the passage of flow.

Regarding the phase separation system (14), the following relationships in the size of their parts are fulfilled:
D_{S}/D_{R}=2.47
D₁=D_{R}
D₂=D_{D}
D₃/D₂=1.71
D₄/D₁=1.64
H_{S}/D_{S}=0.40
H₃/H_{S}=0.94
H₂/H₃=0.35
H₁/H₃=0.60
H₄/H₁=0.55
H₅/H₁=0.39
H_{C}/H_{S}=0.75
H_{B}/H_{S}=0.28

In which D_{S} is the diameter of the upper covering (23), D_{R} is the diameter of the external cylinder (2); D_{D} is the diameter of the internal cylinder (3); D₁ is the upper diameter of the external separator part (16); D₂ is the upper diameter of the internal separator part (15); D₃ is the lower diameter of the internal separator part (15); D₄ is the lower diameter of the external separator part (16); H_{S} is the height of the cylindrical part of the wall of the separator phase (14); H₃ is the height of the cylindrical part of the external separator part (16); H₂ is the height of the conical part of the external separator part (16); H₁ is the height of the upper cylindrical part of the internal separator part (15); H₄ is the height of the conical part of the internal separators part (15); H₅ is the height of the lower cylindrical part of the internal separator part (15); H_{C} is the height of the conical wall of the phase separator (14) wall (14); H_{B} is the height of the ring-shaped gutter (24).

### EXAMPLE 1

A test of ferrous solution (25 g Fe(II)/L) oxidization and production of oxidizing microorganisms of the Leptospirillum genre was carried out in an air-lift bioreactor with a total volume of 256 L, in order to prove the capabilities of the air-lift bioreactor of the present invention. The protocol used in this test was the following.

The bioreactor used had a total volume of 256 L, of which the reaction volume was 131 L, and the phase separator volume was 125 L.

The culture media used in the propagation of the microorganisms had the following composition: 125 g FeSO₄/L, 0.25 g (NH₄)₂SO₄/L, 0.032 g NaH₂PO₄.H₂O/L, 0.013 g KH₂PO₄/L, 0.025 g MgSO₄.7H₂O/L, 0.005 g CaCl₂/L. The pH of the culture media was adjusted to 1.4.

To start the culture, 230 L of culture media were mixed with 20 L of inoculum carrying microorganisms of the Leptospirillum genre.

The biomass-support contents were 40 g/L, made up mainly by ferric precipitates, and to a lesser degree, of diatomaceous earth.

Air with a flow volume of 80 L/min was supplied to allow the growth of microorganisms in the bioreactor. The temperature of the reactor was controlled at 30° C. The pH in the bioreactor was controlled at a value of 1.4 by adding H₂SO₄.

The bioreactor was operated in continuous mode for 140 hours, and fed with culture media of the indicated composition, at pH 1.2, with a flow volume of 30 L/h. The hydraulic residence time of the solution in the reaction zone was 4.8 h. The bioreactor was equipped with temperature, dissolved oxygen, redox potential and pH sensors.

During the operation of the bioreactor, the growth of microorganisms was monitored by microscopic count using a Petroff-Hausser chamber. The concentration of total iron was determined by atomic absorption. The concentration of iron (II) was spectrophotometrically determined by the o-phenanthroline method or estimated from the measuring of redox potential in the culture.

### RESULTS

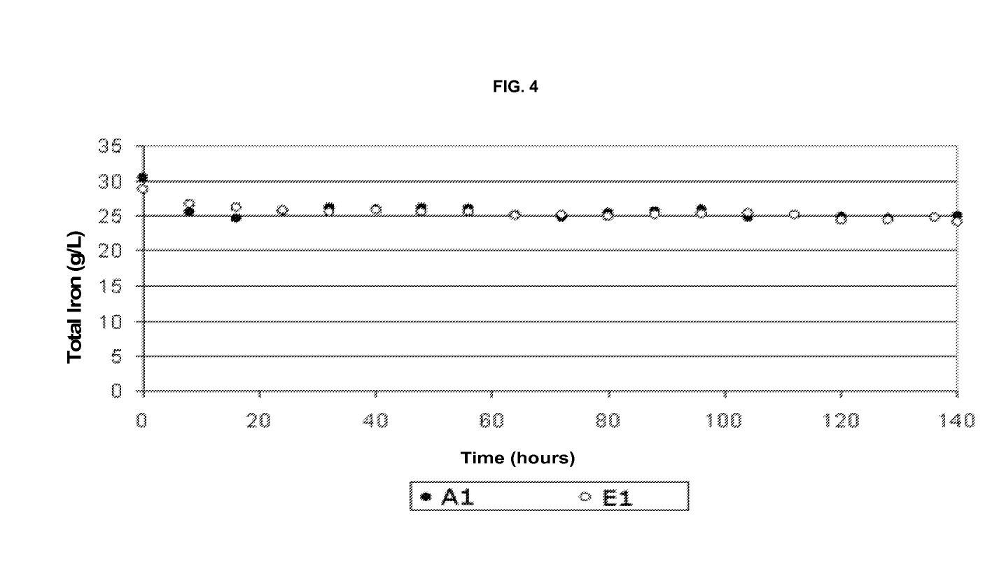

FIG. 4 shows the concentrations of total iron in the bioreactor feed (A1) and effluent (E1) throughout the test. The concentration of total iron in both streams is practically the same, ruling out the precipitation of iron within the equipment during this phase.

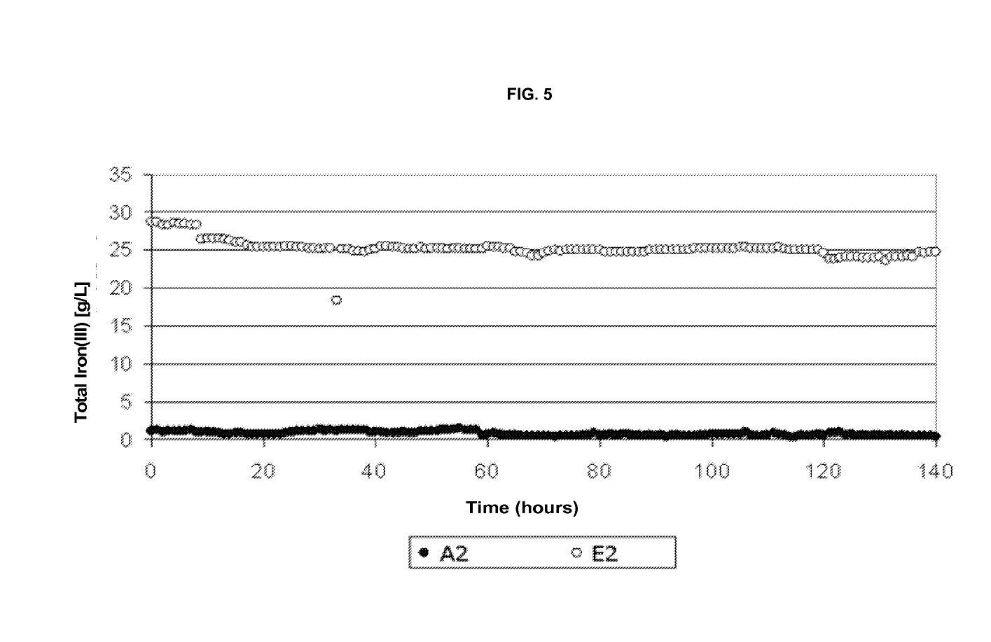

FIG. 5 shows the concentrations of ferric iron in the bioreactor feed currents (A2) and effluent (E2) during the execution of the test, that were estimated based on the contents of total iron in the system, and on the redox potential measurements.

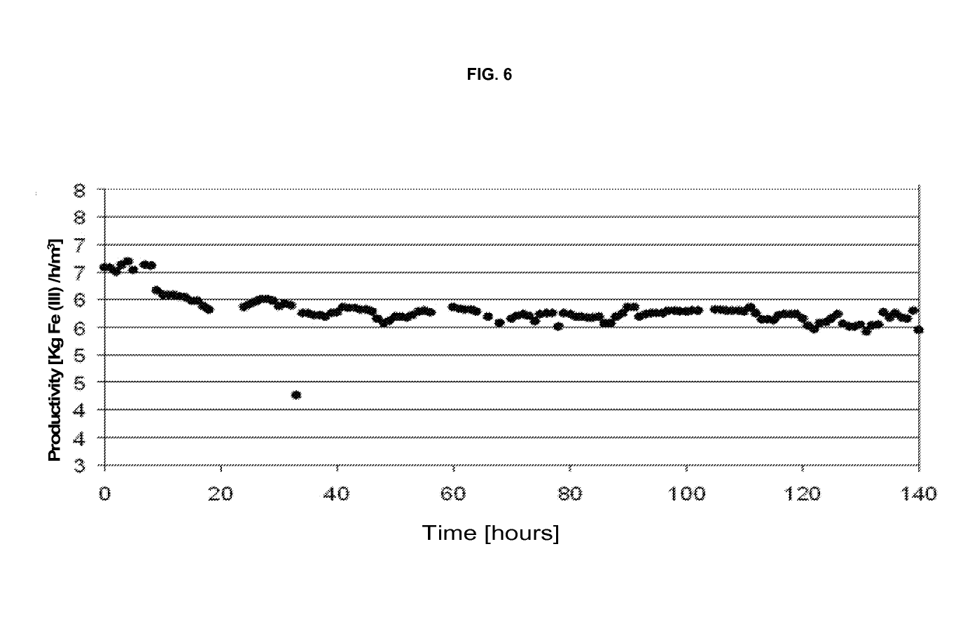

Based on the previous information, the degree of conversion of ferrous ion to ferric ion in the bioreactor during the execution of the test, which was more than 98%, was determined. As we can observe in FIG. 6, productivity of the process iron (III), regarding the reaction volume of 131 L, was 5.8 KgFe(III)/h/m³ during the execution of the test.

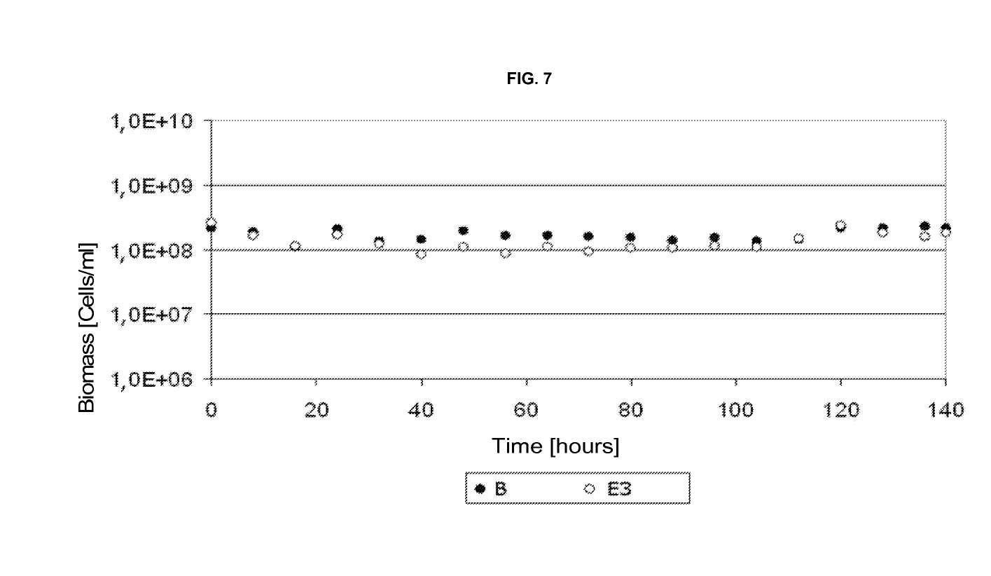

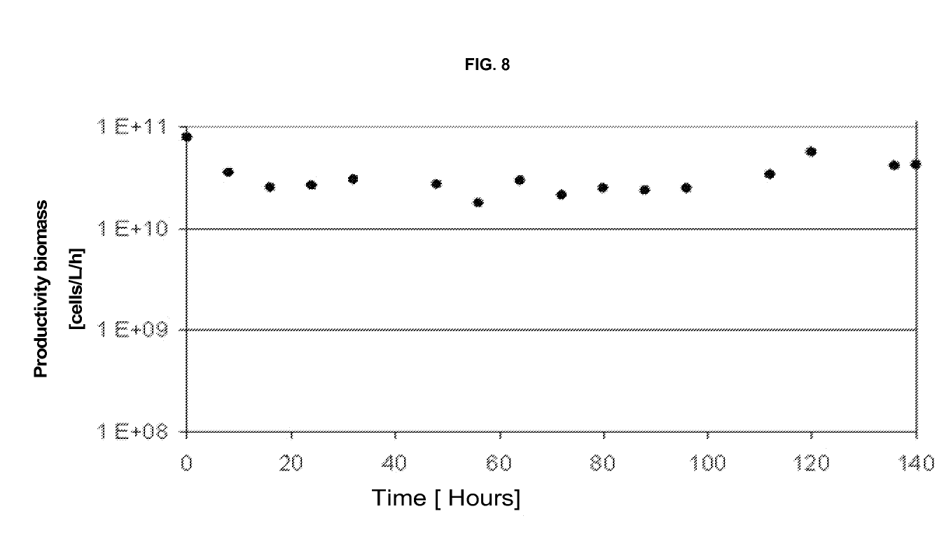

FIG. 7 shows the concentrations of free biomass (identified by count in Petroff-Hausser chamber) in the reaction zone and effluent of the bioreactor, throughout the test. We can observe that the concentration was kept close to 1·10⁸ cells/ml, and the value in the effluent (E3) was slightly lower than the value in the bioreactor (B). As we can observe in FIG. 8, free biomass productivity regarding the reaction volume of 131 L, was close to 3·10¹⁰ cells/L/h.

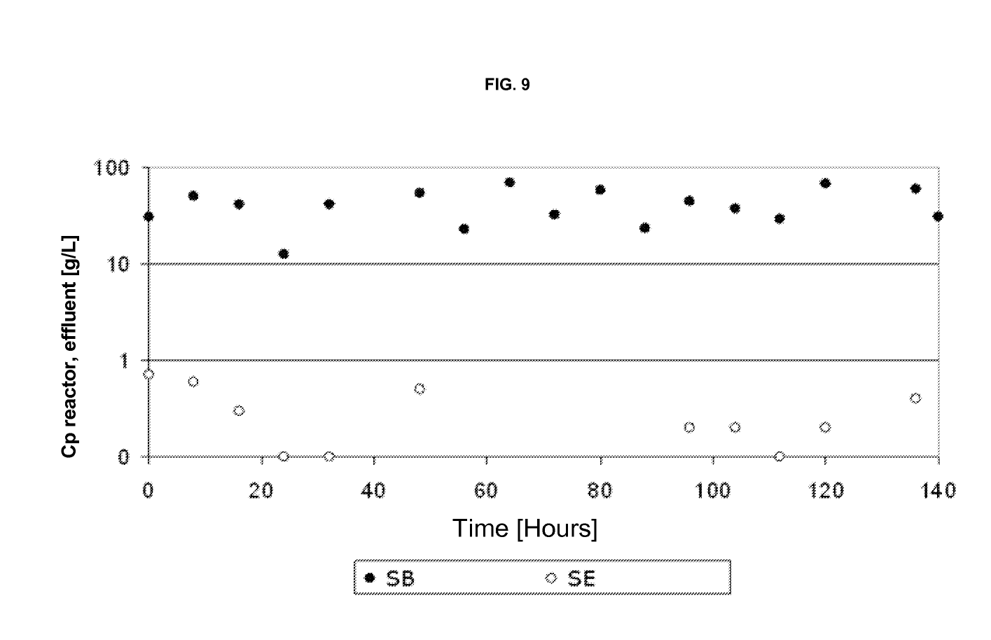

FIG. 9 shows the concentrations of solids (c_{P}) in the bioreactor body (SB) and in its effluents (SE). The content of solids in the effluent is approximately 2 orders of magnitude lower than in the reacting zone, which proves the merits of the phase separator (14) design. Based on the information regarding solid contents in the bioreactor and in the effluent, the efficiency of the phase separator (14) retaining solids (1−c_{P efluente}/c_{P reactor}), which was approximately 99% throughout the test, was determined.

The results show that with the bioreactor previously described, it is possible to have high biomass contents in effluents with less than 5 hours residence time, considering the volume of the reacting zone, which improves the state of the art at total-iron levels of around 25 g/l. Additionally, the design set forth in the present patent application achieves over 99% solids retention within the bioreactor, an important condition for the use of the solutions in the irrigation of belts, heaps and dumps, to avoid clogging-up the sprinklers.

1. A bioreactor for continuous production of bioleaching solutions with high concentrations of microorganisms and ferric ions, for inoculation and irrigation of sulfide-ore bioleaching heaps and dumps. wherein it essentially includes:
a reaction zone, that includes on external cylinder, and an internal cylinder concentric to the external cylinder, which separates the reaction zone into an ascending column (riser) and a descending column (down-comer), an upper recirculation zone from the riser to the down-comer and a lower recirculation zone from the down-comer to the riser;
an inlet for acid;
an inlet for culture media;
a heater in the reaction zone to keep the temperature within a range appropriate for the growth of the microorganisms present;
air injection devices in the lower part of the reaction zone, connected to the corresponding air-input pipe located in the lower part of the equipment;
a discharge outlet for rapid emptying of the bioreactor in the lower recirculation zone;
a phase separator which includes:
a concentric internal separator part that defines the input zone in which the air bubbles of the three phase mixture are trapped and carried towards a de-airing zone and also defines the gaseous separation zone to separate the air bubbles from the three phase mixture.
a concentric external separator part that defines the de-airing zone between its inside and the internal separator part and the external separator part; the zone of conduction of the two phase mixture (solid-liquid) to a solids separation zone;
an exit chimney located in an upper covering of the phase separator and in fluid communication with the de-airing zone,
a ring-shaped gutter to form a zone for the accumulation of the clear liquid that exits the bioreactor,
a cylindrical wall with a dentate rim abutted against the wall of the phase separator marking the limits of the ring-shaped gutter, and
an outlet for collecting the solution concentrated in iron and/or sulfur oxidizing bacteria, and ferric ions.

2. The bioreactor, according to claim 1, wherein the lower part of the external separator part has a conical shape that widens downwards, and is prolonged until it remains at a distance of between 4 and 8 cm from the external conical wall of the lower segment of the phase separator, and the upper part of the external separator part is cylindrical and joined to the upper covering of the bioreactor.

3. The bioreactor, according to claim 1, wherein recirculation of solids from the upper separator to the lower zone occurs without support from a pumping system

4. The bioreactor, according to claim 1, wherein the air-injection devices consist in a set of diffusers of a perforated-pipe type, distributed at a short distance on the lower border of the internal cylinder that separates the riser from the down-comer, in which the diffusers are connected to an air-feeding main with an air inlet.

5. The bioreactor according to claim 3, wherein the diffusers include vents located at both their sides or at a maximum angle of 45° below horizontal.

6. The bioreactor, according to claim 1, wherein the microorganisms are in suspension and immobilized on a solid support whose particle size is within the rage of 30 to 1000 microns.

7. The bioreactor, according to claim 5, wherein the solid support used to immobilize the microorganisms is an inorganic solid such as diatomaceous earth, sulfur, ferric precipitates, copper concentrates, pyrite, or copper ore whose particle size is within the range of 30 to 1000 microns.
